(19) United States
(12) Patent Application Publication
(10) Pub. No.: US 2021/0338004 A1
(43) Pub. Date: Nov. 4, 2021

(54) CANNABINOID-CONTAINING CONSUMER PRODUCT SYSTEMS, APPARATUS AND METHODS

(71) Applicant: HEXO OPERATIONS INC., Ottawa (CA)

(72) Inventors: Max ALSAYAR, Ottawa (CA); Denis KESERIS, Bais d'Urfe (CA); Sebastien ST-LOUIS, Ottawa (CA)

(21) Appl. No.: 17/269,647

(22) PCT Filed: Aug. 20, 2019
(86) PCT No.: PCT/CA2019/051132

§ 371 Date: Feb. 19, 2021

Related U.S. Application Data
(60) Provisional application No. 62/719,966, filed on Aug. 20, 2018.

(51) Int. Cl. A47J 31/40; A47J 31/44; A47J 31/52; A47J 31/46; B65D 85/804

(52) U.S. Cl. CPC A47J 31/407; A47J 31/402; A47J 31/404; A47J 31/4492; B65D 2203/10; A47J 31/461; B65D 85/8058; B65D 2203/06; A47J 31/521

(57) ABSTRACT

The disclosure relates to an apparatus for making cannabinoid-containing beverages. The apparatus comprises at least one interface configured to receive a cartridge containing a cannabinoid-containing substance. The apparatus also comprises at least one valve, in fluid communication with the at least one interface, configured to dispense an amount of the substance from the cartridge. A valve controller is configured to control the valve in order to vary the amount of the substance dispensed by the valve.

### CROSS-REFERENCE TO RELATED APPLICATIONS

This application claims priority from U.S. Provisional Patent Application 62/719,966 filed on Aug. 20, 2018 and hereby incorporated by reference herein.

### FIELD

This disclosure relates generally to cannabinoid-containing consumer products. In particular, the disclosure relates to systems, apparatus, methods and formulations for preparing cannabinoid-containing liquid and powder additives for beverages or other edibles, apparatus for delivering or consuming cannabinoid-containing beverages, and formulations of cannabinoid-containing liquid and powder additives for use in beverages or other edibles.

### BACKGROUND

While the market for cannabinoid-containing consumer products is gaining momentum, historically, the clandestine nature of the cannabis industry has largely suppressed innovation, and led to a market characterised by unsophisticated product development activity, as well underdeveloped consumer product safety and characterisation standards.

For examples, traditional product descriptors, such as indica, hybrid and sativa, often have very little to do with a particular product's actual chemistry. As cannabinoid-containing products reach a growing number of mainstream consumers, these inadequacies will have significant adverse effects on consumers' and legislators' perceptions of the cannabis-related consumer product market.

For these and other reasons, there is a need for improvements in systems, apparatus, methods and formulations for preparing cannabinoid-containing liquids and powder additives for beverages or other edibles, apparatus for delivering or consuming cannabinoid-containing beverages, and formulations of cannabinoid-containing liquid and powder additives for use in beverages or other edibles that will satisfy a growing demand and need for sophisticated consumption technologies that will afford consumers precise, consistent and tailored experiences and enable technology-driven and data-driven standardization of cannabis-based consumer products.

### SUMMARY

The present disclosure generally relates to smart devices for preparing water-soluble cannabis-compound-based beverages. These devices are configured to accept and make use of consumable cartridges containing liquid-soluble cannabinoid-containing substances and/or other flavoring additives. As defined herein, a cannabinoid-containing substance can take the form of a liquid, slurry or a fluidized solid (e.g. powder). The disclosure also relates to effervescent tablets containing liquid-soluble cannabinoid-containing substances and apparatus for dispensing these.

According to an aspect, the disclosure relates to an apparatus for making cannabinoid- containing beverages. The apparatus comprises at least one interface configured to receive a cartridge containing a cannabinoid-containing substance. The apparatus also comprises at least one valve, in fluid communication with the at least one interface, configured to dispense an amount of the substance from the cartridge and a valve controller configured to control the valve in order to vary the amount of the substance dispensed by the valve.

According to another aspect, the disclosure relates to a computer-implemented method of controlling an apparatus for making cannabinoid-containing beverages, the apparatus comprising at least one interface configured to receive a cartridge containing a cannabinoid-containing substance and a cartridge tag, and at least one cartridge tag reader configured to read the cartridge tag, the cartridge tag being configured to convey cartridge information relating to the cartridge. The method comprises the steps of reading the cartridge information from the cartridge tag and disabling the apparatus if the cartridge information does not correspond to predetermined cartridge information.

According to yet another aspect, the disclosure relates to a computer-implemented method of controlling an apparatus for making cannabinoid-containing beverages, the apparatus comprising at least one interface configured to receive a cartridge containing a cannabinoid-containing substance and at least one volume sensor configured to generate content information conveying information relating to the quantity of substance remaining in the cartridge. The method comprises the steps of receiving content information from the volume sensor and extracting the quantity of substance remaining in the cartridge from the content information. The method also comprises the steps of determining whether the quantity of substance remaining in the cartridge is below a threshold and generating an alert signal if the quantity of substance remaining in the cartridge is below the threshold.

According to yet another aspect, the disclosure relates a computer-implemented method of controlling the aforementioned. The method comprises the steps of connecting to a formulation server configured to provide information indicative of a plurality of formulations of different combinations of cannabinoid-containing substances and selecting a formulation from among the plurality of formulations. The method also comprises the steps of receiving the information indicative of the selected formulation at the mobile device and receiving the information indicative of the selected formulation at the apparatus. The method also comprises the steps of configuring the valve controller in accordance with the selected formulation.

According to yet another aspect, the disclosure relates to a cartridge for use with the aforementioned apparatus. The cartridge comprises an amount of a cannabinoid-containing substance and a cartridge tag configured to convey cartridge information relating to the cartridge.

According to yet another aspect, the disclosure relates to an effervescent tablet that comprises at least one liquid-soluble or partially liquid-soluble cannabinoid-containing substance.

According to yet another aspect, the disclosure relates to a consumer product that comprises a connected array of individually-wrapped effervescent tablets, each containing a liquid-soluble or partially liquid-soluble cannabinoid-containing substance.

According to yet another aspect, the disclosure relates to an apparatus for dispensing effervescent tablets comprising at least one liquid-soluble or partially liquid-soluble cannabinoid-containing substance. The apparatus comprises a plurality of chambers, each configured to hold a plurality of effervescent tablets. Each chamber comprises a dispensing end have an opening configured to allow an effervescent tablet to pass therethrough, a mechanical biasing means for biasing the effervescent tablets towards the dispensing end, and a dispensing actuator configured to move one effervescent tablet at least partially through the opening.

According to yet another aspect, the disclosure relates to an apparatus for preparing a cannabis-containing drink. The apparatus comprises a first source of cannabinoid material, a second source of cannabinoid material, and dispensing means in communication with the first source of cannabinoid material and with the second source of cannabinoid material for combining a first quantity of cannabinoid material from the first source with a second quantity of cannabinoid material from the second source into a drink.

### BRIEF DESCRIPTION OF THE DRAWINGS

A detailed description of examples is provided below, with reference to the following drawings, in which:

FIG. 1 shows a diagram of an example device for preparing and dispensing cannabinoid-containing beverages;

FIG. 2 shows a functional schematic diagram of the example device of FIG. 1 for preparing and dispensing cannabinoid-containing beverages;

FIG. 3 shows an example cartridge for use with the example devices of FIG. 1, FIG. 5 and FIG. 8 for preparing and dispensing cannabinoid-containing beverages;

FIG. 4 shows an example Graphical User Interface (GUI) of the example device of FIG. 1 for preparing and dispensing cannabinoid-containing beverages;

FIG. 5 shows an example hand-held device for preparing cannabinoid-containing beverages;

FIG. 6 shows a functional schematic diagram of the example hand-held device of FIG. 5 for preparing and dispensing cannabinoid-containing beverages;

FIG. 7 shows an example Graphical User Interface (GUI) of a communication device configured to control devices for preparing cannabinoid-containing beverages;

FIG. 8 shows a diagram of an example device for preparing cannabinoid-containing beverages;

FIG. 9 shows a flowchart representing an example method of validating a cartridge for use with an example device for preparing cannabinoid-containing beverages;

FIG. 10 shows a flowchart representing an example method of automatically reordering a cartridge for use with an example device for preparing cannabinoid-containing beverages;

FIG. 11 shows a flowchart representing an example method of downloading a formulation recipe for use with an example device for preparing cannabinoid-containing beverages;

FIG. 12 shows a flowchart representing an example method of uploading a formulation recipe for use with an example device for preparing cannabinoid-containing beverages;

FIG. 13 shows a high-level network diagram of an example data-communication system configured to be used with example devices for preparing cannabinoid-containing beverages;

FIG. 14 shows an example effervescent tablet for creating a cannabinoid-containing beverage;

FIG. 15 shows an example of a consumer product for the safe distribution and sale of a plurality of example effervescent tablets; and

FIG. 16 shows an example of a consumer product for the safe distribution and sale of a plurality of example effervescent tablets.

These and other aspects of this disclosure will now become apparent to those of ordinary skill in the art upon review of a description of embodiments that follows in conjunction with the accompanying drawings.

### DETAILED DESCRIPTION

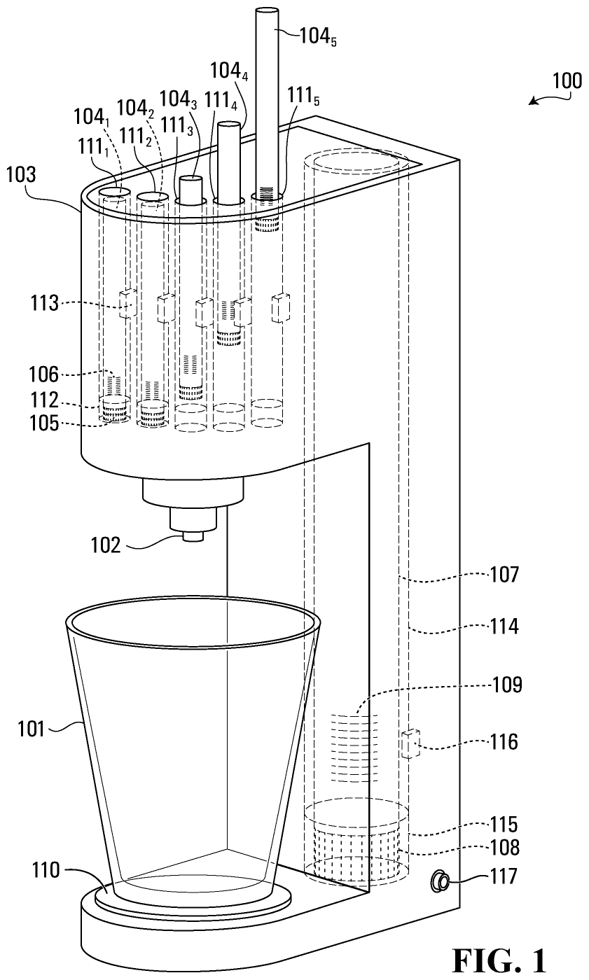

FIG. 1 shows a diagram of an example device 100 for preparing and/or dispensing liquid formulations of cannabinoid-containing beverages. In some embodiments, the device is configured to dispense cannabinoid-containing substances to be added to existing beverages found in beverage receptacle 101. In some embodiments, the device 100 is configured to dispense cannabinoid-containing beverages into beverage receptacle 101. In yet other embodiments, the device 100 is configured to dispense cannabinoid-containing substances that are to be added to existing beverages found in beverage receptacle 101 and to dispense cannabinoid-containing beverages into beverage receptacle 101. As herein defined, the term substance includes but is not limited to liquids, powders, oils, resins and effervescent tablets. In some embodiments, the substance is an emulsified powder.

In some embodiments, the device 100 comprises a base 110 configured to support a receptacle 101 for receiving a beverage. In some embodiments, the device 100 also includes a dispensing head 103 configured to dispense a cannabinoid-containing beverage or a cannabinoid-containing concentrate for adding to a beverage. The dispensing head comprises a plurality of slots 111, configured to receive a plurality of cartridges 104₁₋₅. In the embodiment shown in FIG. 1, the device 100 comprises five slots 111ₓ, though in other embodiments, the device 100 can include any number of slots 111ₓ, for example ten slots 111ₓ or twenty slots 111ₓ.

With reference to slot 111₁, in some embodiments, each slot 111ₓ comprises a device interface 112 for connecting to a corresponding cartridge interface 105 forming part of cartridge 104₁. Interfaces 112 and 105 allow for liquid or powder to flow from the cartridge 104₁, into the device 100. In some embodiments, interfaces 112 and 105 allow for securing cartridge 104₁ to the device 100. In some embodiments, interfaces 112 and 105 also allow for electronic, chemical, or electromechanically communication between functional elements of cartridge 104₁ and the device 100. Each slot 111ₓ also comprises a cartridge tag reader 113 configured to read a cartridge tag 106 included on (or in) each cartridge 104ₓ. The dispensing head 103 also includes a spout 102 in controlled fluid communication with the device interface of each slot 111ₓ.

In some embodiments, the device 100 also includes a further slot 114 for receiving cartridge 107. In some embodiments, cartridge 107 is larger than cartridges 104₁₋₅ and is configured to contain and dispense a base liquid for a beverage. Some examples of base liquids for a beverage include but are not limited to drinking water, milk (both diary and non-diary), juice, a smoothie, coffee or a caffeinated beverage, tea, herbal tea, a cocoa beverage, any type of carbonated drink, an energy drink, a drinkable yogurt, a fermented beverage, or an alcoholic drink. Cartridge 107 may contained pressurized liquid or not. In other embodiments, cartridge 107 includes a pressurized gas canister configured to carbonate or nitrogenate a base liquid for a beverage which is provided, for example, via fluid connector fitting 117, which is also in fluid communication with spout 102. In some embodiments, the device 100 also include a further device interface 115 in controlled fluid communication with the spout 102. The device interface 115 is configured to receive a cartridge interface 108 forming part of a cartridge 107, as will be described in more detail below. In some embodiments, slot 114 also includes a cartridge tag reader 116 configured to read a cartridge tag 109 included on or in cartridge 107.

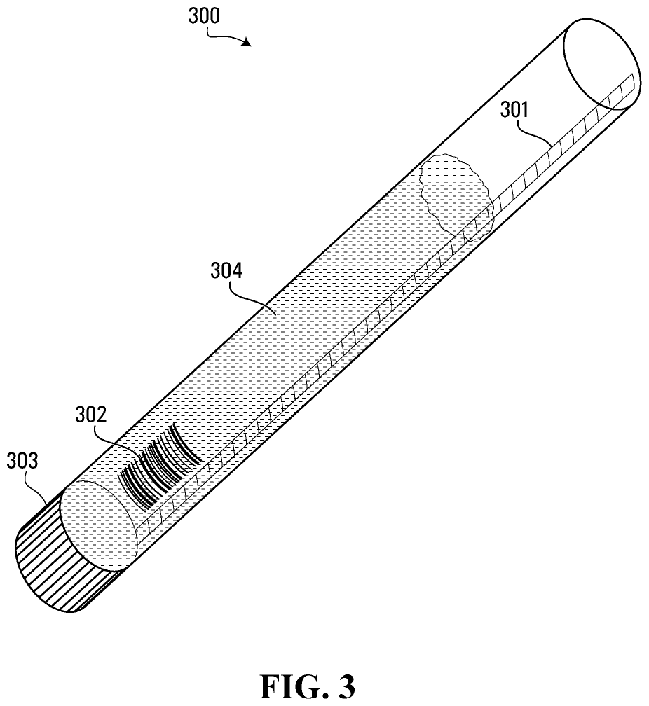

FIG. 3 shows an example cartridge 300 for use with an example device for preparing and dispensing cannabinoid-containing beverages. In some examples the cartridge 300 comprises a cartridge interface 303 and a liquid or powder reservoir 304. The liquid or powder reservoir 304 can be made of any suitable material, including, but not limited to, glass and plastic. In some examples, the cartridge interface 303 is configured to mechanically cooperate with a corresponding device interface to form a coupling for allowing powder or liquid to substantially freely flow from the liquid or powder reservoir 304 into device 100. In some examples, the cartridge interface 303 and the device interface are configured to interlock mechanically. In some examples, fluid communication from the reservoir 304 holding the liquid or powder concentrate, through the cartridge interface 303, and into the device via the device interface is enabled by mechanical interlocking between the cartridge interface 303 and the device interface.

In some examples, the cartridge interface 303 is configured to electronically cooperate with a corresponding device interface (e.g. device interface 112) configured to receive the cartridge interface 303. In some examples, the cartridge interface 303 can comprise an RFID tag configured to convey information related to the cartridge. In other examples, the cartridge interface 303 comprises other mechanical or electronic means for conveying information related to the cartridge.

In some embodiments, the cartridge interface 303 is configured to be child resistant until it is engaged with one of the device interfaces 112, 511, 808 of one of devices 100, 500, 800. The child resistance can be provided mechanically or electronically. For example, flow of the powder or liquid from the reservoir 304 could be prohibited until mechanical or electrical enablement of a valve actuated by coupling with one of the device interfaces 112, 511, 808 of one of devices 100, 500, 800.

Examples of information relating to the cartridge include, but are not limited to, the contents of the cartridge 300 (e.g. the identity of any cannabinoids present in the contents of the cartridge, as well as the amount by volume of the same), the identity of the manufacturer, the identity of the distributor and/or the identity of the vendor of the cartridge 300, or information relating to the shelf life of the contents of the cartridge 300.

In some embodiments, the cartridge 300 comprises a cartridge tag 302 for conveying information relating to the cartridge. In the non-limiting example of FIG. 3, the cartridge tag 302 is a barcode. In some embodiments, a barcode required to comply with regulatory requirements relating to cannabis-based product packaging is the cartridge tag 302 used to identify the cartridge. Other suitable cartridge tags 302 include, but are not limited to, Quick Response (QR™) codes, or other suitable matrix barcodes, Radio Frequency Identification (RFID) tags, or any other suitable tag able to convey the aforementioned information relating to the cartridge 300.

In some embodiments, cartridge 300 comprises a volume sensor 301 configured to measure the remaining quantity of liquid or powder present in the reservoir. Examples of volume sensors 301 include but are not limited to load cells, strain gauges, capacitance level sensors, ultrasonic level sensors, optical level switches or conductive sensors. The volume sensors 301 are further configured to emit a signal indicative of the remaining quantity of liquid or powder present in the reservoir, which signal can be communicated to the device 100, 500, 800, by way of the cartridge/device interfaces. Information relating to the remaining quantity of liquid or powder present in the reservoir can therefore be saved to memory 216 each time a composition is dispensed. Alternatively, the information relating to the remaining quantity of liquid or powder present in the reservoir can be saved to memory 216 at set intervals of time.

In other embodiments, a signal indicative of the remaining quantity of liquid or powder present in the reservoir can be generated by a volume sensor forming part of the device. In some examples, each slot 111ₓ comprises a volume sensor for determining the remaining quantity of liquid or powder present in the reservoir associated with the cartridge inserted in the respective slot 111ₓ. In some embodiments, the volume sensor associated with the each slot 111ₓ is configured to optically determine the level of substance remaining in the reservoir associated with the cartridge inserted in the respective slot 111ₓ.

In some examples, each of cartridges 104₁, 104₂, 104₃, 104₄, 104₅ comprises a cannabis-based additive concentrate, cannabis-based flavouring concentrate, other flavoring concentrate or any other additive suitable to produce a cannabis-based liquid formulation. Such cannabis-based additives include, but are not limited to, those described herein. In some examples, cannabis-based additives include cannabinoid-based liquid or powder concentrates including one or more substantially isolated cannabinoid made water-soluble, and configured to produce a cannabis-based liquid formulation when dissolved in a water-containing beverage. In some examples, the one or more substantially isolated cannabinoids include tertrahydrocannabinol (THC), cannabidiol (CBD), and/or any other major or minor cannabinoid. In some examples, the cannabis-based additives include terpene-based additives including one or more substantially isolated cannabis terpenes made water-soluble. In some examples, the substantially isolated cannabis terpenes include limonene, linalool, caryophyllene, camphene, and/or any other cannabis terpene. In some examples, a cartridge 104ₓ can contain any other natural or synthetic flavoring suitable for use in beverages. In some examples, a cartridge 104ₓ can contain any other natural or synthetic additive suitable for use in beverages.

As used herein, the term “liquid formulation” means a formulation that flows freely or under pressure but is of a constant volume. A liquid formulation provided herein may be a liquid additive that is suitable to be added to a beverage. A liquid formulation provided herein may be a beverage. In some embodiments, the liquid formulation is a beverage, such as drinking water, milk (both diary and non-diary), juice, a smoothie, coffee or a caffeinated beverage, tea, herbal tea, a cocoa beverage, a carbonated drink, an energy drink, a drinkable yogurt, a fermented beverage, or an alcoholic drink.

In some embodiments, the liquid formulation is a carbonated drink. The carbonated drink may have zero calories. In some embodiments, the liquid formulation is an alcoholic drink, including but not limited to, beer, lager, cider, spirits, wine/fortified wine, and cocktails.

As used herein, the term “cannabinoid” is generally understood to include any chemical compound that acts upon a cannabinoid receptor. Cannabinoids may include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A liquid formulation provided herein may comprise a cannabinoid in its pure or isolated form or a source material comprising the cannabinoid. Examples of source materials comprising cannabinoids include, but are not limited to, cannabis or hemp plant material (e.g, flowers, seeds, trichomes, and kief), milled cannabis or hemp plant material, s extracts obtained from cannabis or hemp plant material (e.g., resins, waxes and concentrates), and distilled extracts or kief. In some embodiments, pure or isolated cannabinoids and/or source materials comprising cannabinoids may be combined with water, lipids, hydrocarbons (e.g., butane), ethanol, acetone, isopropanol, or mixtures thereof.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some embodiments, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-l-yl)-5-pentyl-1,3-benzenediol); (2) Δ4-cannabidiol (2-(6-isopropenyl-3-methyl-4-cyclohexen-l-yl)-5-pentyl-l,3-benzenediol); (3) Δ3-cannabidiol (2-(6-isopropenyl-3-methyl-3-cyclohexen-l-yl)-5-pentyl-l,3-benzenediol); (4) Δ3,7-cannabidiol (2-(6-isopropenyl-3-methylenecyclohex-l-yl)-5-pentyl-l,3-benzenediol); (5) Δ2-cannabidiol (2-(6-isopropenyl-3-methyl-2-cyclohexen-l-yl)-5-pentyl-l,3-benzenediol); (6) Δ1-cannabidiol (2-(6-isopropenyl-3-methyl-l-cyclohexen-l-yl)-5-pentyl-l,3-benzenediol); and (7) Δ6-cannabidiol (2-(6-isopropenyl-3-methyl-6-cyclohexen-l-yl)-5-pentyl-l,3-benzenediol).

In some embodiments, the cannabinoid is a mixture of tetrahydrocannabinol (THC) and cannabidiol (CBD). The w/w ratio of THC to CBD in the liquid formulation may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some embodiments, the formulation may include products of cannabinoid metabolism, including 11-hydroxy-Δ9-tetrahydrocannabinol (11-OH-THC).

In some embodiments, a liquid formulation provided herein may comprise one or more encapsulating agents that form a microencapsulation system with the cannabinoid in the liquid formulation. Encapsulating agents may act as a coating material that completely surrounds the cannabinoid, or as a homogeneous or heterogeneous matrix in which the cannabinoid is embedded.

The cannabinoid may be solubilized in a carrier oil or solvent prior to microencapsulation. Carrier oils and solvents suitable for cannabinoids include, but are not limited to, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g. caprylic/capric glycerides), ethanol, acetone, isopropanol, hydrocarbons and any combination thereof.

In some embodiments, the one or more encapsulating agents are film-forming natural or synthetic biopolymers, including proteins, carbohydrates, lipids, fats, and gums, or one or more small-molecule surfactants, or any combination thereof. In some embodiments, the one or more encapsulating agents may be gum arabic; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens (polysorbates) such as Tween 20 (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale®) and components thereof; sorbitan esters (Spans) such as Span 20 (sorbitan monolaurate), Span 40 (sorbitan monopalmitate), Span 60 (sorbitan monostearate), Span 80 (sorbitan monooleate); amphiphilic block copolymers; cholesterol; egg yolk-and soy-derived phosphatidylcholines; cyclodextrins such as 2-hydroxypropyl-β-cyclodextrin; lecithin; or any combination thereof.

Microencapsulation techniques may include emulsification and nanoemulsification techniques, mixing, homogenization, injection, spray drying, spray cooling, spray chilling, freeze-drying, air suspension coating, fluidized-bed extrusion, centrifugal extrusion, coacervation, rotational suspension separation, cocrystallization, liposome entrapment, interfacial polymerization, molecular inclusion, microfluidization, ultrasonication, physical adsorption, complex formation, nanosized self-assembly, or any combination thereof. The microencapsulation process may be assisted or accelerated by the application of heat, e.g., through microwave irradiation. Mixing may be modelled using idealized chemical reactors, which may include, but are not limited to, batch reactors, continuous stirred-tank reactors, and plug flow reactors.

Microencapsulation systems may include emulsions, nanoemulsions, micelles, solid lipid nanoparticles, nanostructured lipid carriers, liposomes, nanoliposomes, niosomes, polymer particles, or hydrogel particles.

In some examples, each cartridge 104ₓ comprises a cartridge tag 106. The cartridge tag 106 is configured to convey information relating to the cartridge 104ₓ to device 100. Examples of information relating to the cartridge include, but are not limited to, the contents of the cartridge 104ₓ, the manufacturer, distributor and/or vendor of the cartridge 104ₓ, information relating to the shelf life of the contents of the cartridge 104ₓ.

In some examples, the cartridge tag 106 is a barcode and each slot 111ₓ includes a barcode reader 113 configured to read the barcode when a cartridge 104ₓ is inserted in the device slot 111ₓ, or thereafter. Other suitable cartridge tags 106 include, but are not limited to, Quick Response (QR™) codes, or other suitable matrix barcodes, Radio Frequency Identification (RFID) tags, or any other suitable tag able to convey the aforementioned information relating to the cartridge 104ₓ. Similarly, other suitable cartridge tag readers 113 include, but are not limited to, Quick Response (QR™) code readers, or other suitable matrix barcode readers, Radio Frequency Identification (RFID) tag readers, or any other suitable tag readers able to read tags configured to convey the aforementioned information relating to the cartridge 104ₓ.

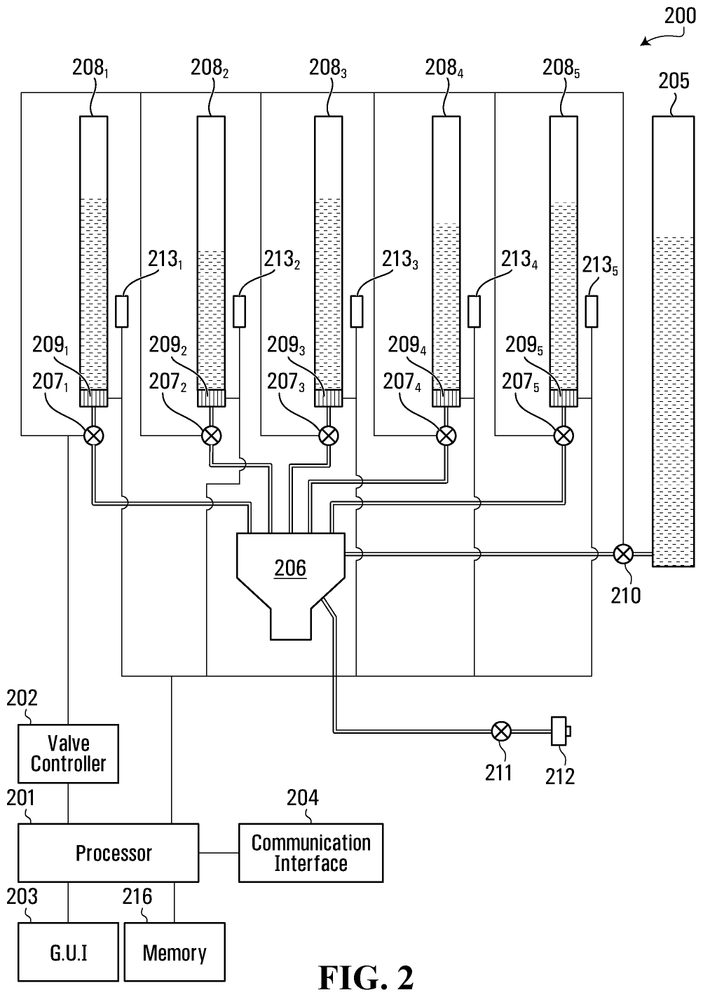

Aspects of the operation of device 100 will now be described with particular reference to FIG. 2, which shows a schematic diagram of an example device for preparing and dispensing cannabinoid-containing beverages. The device 200 comprises a plurality of couplings 209₁₋₅ configured to allow free fluid communication of powders or liquids contained in cartridges 208₁₋₅ into respective metering valves 207₁₋₅. In some embodiments, each coupling 209ₓ consists of a device interface 112 operably engaged with a cartridge interface 105 to allow substantially unobstructed flow of powders or liquids contained in cartridges 208₁₋₅ into respective metering valves 207₁₋₅.

Device 100 also comprises a processor 201 operably connected to a valve controller 202, a communication interface 204, a memory 216 and a Graphical User Interface (GUI) 203. The valve controller 202 is operably connected to metering valves 207₁₋₅. In some embodiments, two or more elements of the processor 201, the valve controller 202 and the communication interface 204 may be implemented by devices that are physically distinct from one another and may be connected to one another via a bus (e.g., one or more electrical conductors or any other suitable bus) or via a communication link which may be wired. In other embodiments, two or more elements of the processor 201, the valve controller 202 and the communication interface 204 may be implemented by a single integrated device. Moreover, in some embodiments, two or more elements of the processor 201, the valve controller 202, the metering valves 207₁₋₅, the cartridge tag readers 213ₓ and the communication interface 204 may be connected to one another via a bus (e.g., one or more electrical conductors or any other suitable bus) or via a communication link which may be wired.

In some embodiments, the device 100 comprises multiple metering valves 207₁₋₅ operable to provide electronically controlled accurate and stable flow rates into spout chamber 206. The flow rate and flow duration of each metering valve 207ₓ is controlled by valve controller 202, using instructions received from processor 201. In other embodiments, metering valves 207₁₋₅ can be replaced by any suitable device configured to precisely meter out (or otherwise dispense with substantial precision) the contents of the cartridges 208₁₋₅ into the spout chamber 206 under control of the valve controller 202, or other equivalent controller.

In some embodiments, the device 100 comprises cartridge tag readers 213₁₋₅ for each cartridge 208₁₋₅, respectively. In some examples, each cartridge 208ₓ comprises a cartridge tag (not shown in FIG. 2). As described above, the cartridge tag is configured to convey information relating to the cartridge 208ₓ to device 200. Examples of information relating to the cartridge include, but are not limited to, the contents of the cartridge 208ₓ, the manufacturer, distributor and/or vendor of the cartridge 208ₓ, information relating to the shelf life of the contents of the cartridge 208ₓ. Once read by a cartridge tag reader 213ₓ, the information relating to the cartridge can be stored in the memory for later retrieval by, for example, the processor 201.

In some examples, each cartridge tag reader 213ₓ includes a barcode reader configured to read a barcode contained on a cartridge 208ₓ when the cartridge 208ₓ is inserted into the device. In some embodiments, the barcode required to comply with regulatory requirements relating to cannabis-based product packaging is the barcode used to identify the cartridge. Other suitable cartridge tags include, but are not limited to, Quick Response (QR™) codes, or other suitable matrix barcodes, Radio Frequency Identification (RFID) tags, or any other suitable tag able to convey the aforementioned information relating to the cartridge 208ₓ. Similarly, other suitable cartridge tag readers 213 include, but are not limited to, Quick Response (QR™) code readers, or other suitable matrix barcode readers, Radio Frequency Identification (RFID) tag readers, or any other suitable tag readers able to read tags configured to convey the aforementioned information relating to the cartridge 208ₓ.

In some embodiments, once the cartridge tag reader 213 reads the information relating to the cartridge 208ₓ from the cartridge tag, the information is sent to processor 201. Processor 201 is configured to use the information relating to the cartridge in a number of ways, as will be described in more detail below.

In some embodiments, the device 200 also includes a further cartridge 205. In some embodiments, cartridge 205 is larger than cartridges 208₁₋₅ and is configured to contain and dispense a base liquid for a beverage. Some examples of base liquids for a beverage include, but are not limited to, drinking water, milk (both diary and non-diary), juice, a smoothie, coffee or a caffeinated beverage, tea, herbal tea, a cocoa beverage, any type of carbonated drink, an energy drink, a drinkable yogurt, a fermented beverage, or an alcoholic drink. Cartridge 205 may contained pressurized liquid or unpressurized liquid. In other embodiments, cartridge 205 includes a pressurized gas canister configured to carbonate or nitrogenate a base liquid for a beverage which is provided, for example, via fluid connector fitting 212, which is also in fluid communication with spout chamber 206. In some embodiments, the device 100 also include a further device interface 115 in controlled fluid communication with the spout 102. The quantity of liquid or gas introduced into the spout chamber 206 from cartridge 205 is controlled by metering valve 210, which is configured to be controlled by valve controller 202. Similarly, the quantity of liquid or gas introduced into the spout chamber 206 from fluid connector 212 is controlled by metering valve 211, which is also configured to be controlled by valve controller 202.

In some embodiments, the spout chamber 206 is configured to mix any powder, liquid and gas dispensed from the metering valves 207ₓ, 210 and 211 before dispensing the resulting mixture into, for example, beverage receptacle 101. In other embodiments, the spout chamber 206 is configured to directly dispense any powder, liquid and gas dispensed from the metering valves 207ₓ, 210 and 211 into the beverage receptacle 101. The aforementioned mixing and/or dispensing steps can be controlled by processor 201.

Processor 201 is configured to generally control operations of the device based on information received from the communication interface 204 and/or the GUI 203. Accordingly, the processor 201 is configured to receive and send information from and to the communication interface 204 and the GUI 203. Moreover, processor 201 is configured to receive information related to the cartridges received form cartridge tag readers 213₁₋₅, as well as information relating to the remaining volume of liquid or powder remaining in the cartridges 208₁₋₅ from the volume sensor 301 in each cartridge 208₁₋₅, by way of couplings 209₁₋₅. Processor 201 is configured to communicate with each metering valve 207₁₋₅, 210 and 211 in order to precisely control the amount of liquid or powder that is dispensed from each metering valve 207₁₋₅, 210 and 211, by way of valve controller 202.

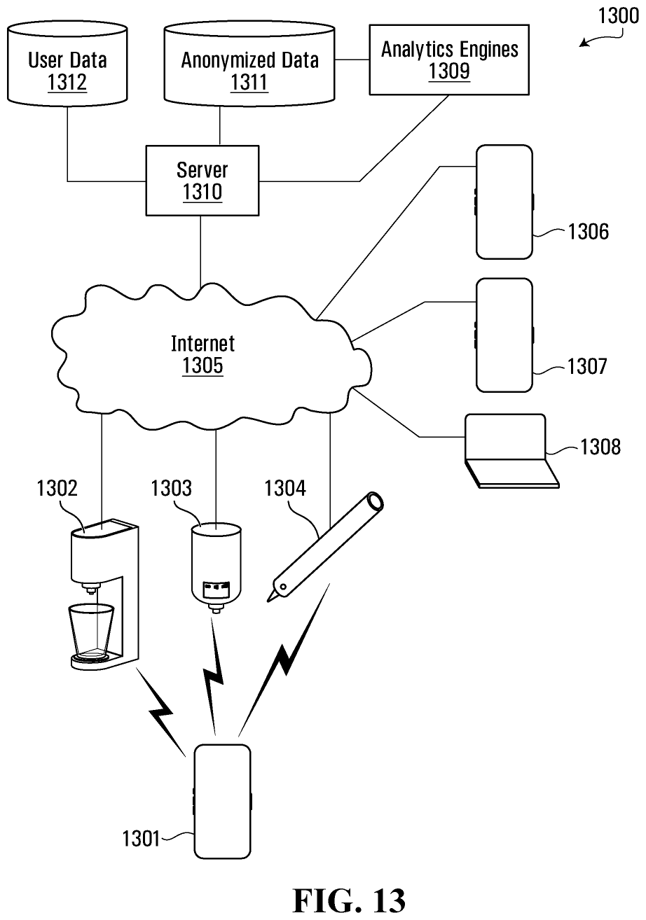

In some examples, the device also comprises a communication interface 204 for communicating with other devices. As shown in FIG. 13, in some examples, the communication interface 204 of the device 1302 may interact with other devices over a communication link, which may be wireless, wired, or partly wireless and partly wired (e.g., Bluetooth or other short-range or near-field wireless connection, WiFi or other wireless LAN, WiMAX or other wireless WAN, cellular, Universal Serial Bus (USB), etc.). In some examples, the other devices can include a smartphone 1301, 1306, 1307 or other wireless phone; a tablet computer 1308; a head-mounted display, smartwatch or other device; or any other communication device carried, worn or otherwise associated with a user. In some examples, the other device can include a server 1310 or other computing entity (e.g., implementing a website) associated with a manufacturer, distributor or vendor of the cartridges. In some examples, such as where the other device is a smartphone, tablet, head-mounted display, smartwatch, or other communication device carried or worn by the user, communication between the other device 1301 and the communication interface of devices 1302, 1303, 1304 may be direct, i.e., without any intermediate device. For instance, in some embodiments, this can be achieved by pairing (e.g., Bluetooth pairing) the device 1302, 1303, 1304 and the other device 1301.

In other cases, such as where the other device 1306, 1307, 1308 is remote from the device 1302, 1303, 1304, communication between the other device 1306, 1307, 1308 and the device 1302, 1303, 1304 may be indirect, e.g., through one or more networks and/or one or more additional communication devices. For example, in some embodiments, the device 1302, 1303, 1304 may communicate with a WiFi hotspot or cellular base station, which may provide access to a service provider and ultimately the Internet 1305 or another network, thereby allowing the another device 1306, 1307, 1308 and the device 1302, 1303, 1304 to communicate. As another example, in some embodiments, communication between the device 1302, 1303, 1304 and another device 1306, 1307, 1308 may take place through a smartphone, tablet, head-mounted display, smartwatch, or other communication device which is carried or worn by the user of the device 1302, 1303, 1304 and which itself may have established communication with a WiFi hotspot or cellular base station. In some embodiments, an application (“app”, i.e., software) may be installed on a mobile device 1301 to interact with the device 1302, 1303, 1304, as described in below.

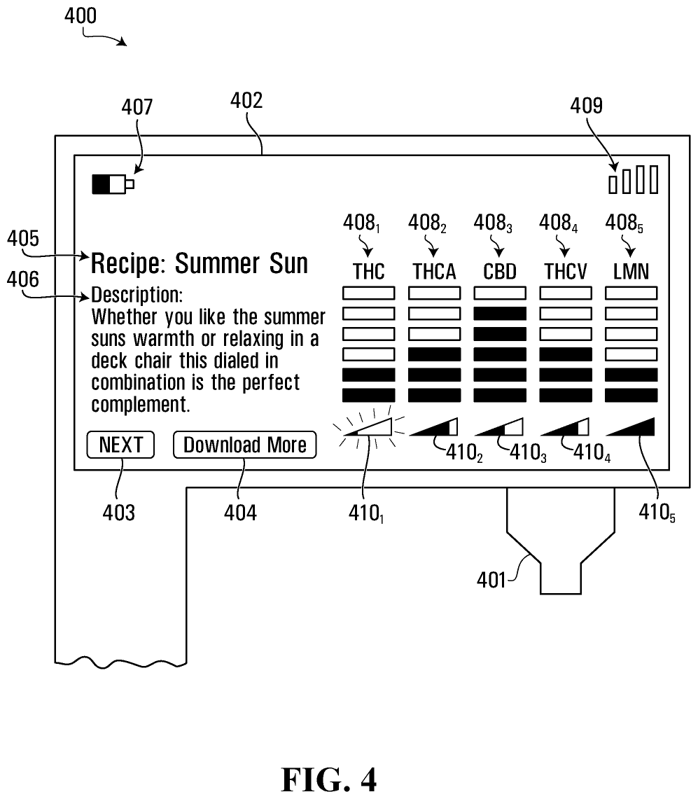

In some embodiments, the device 100, 200, 1302 further comprises a graphical user interface (GUI) 203, 400. An example GUI 400 is shown in FIG. 4. In some embodiments, the device 100 is battery operated, and the GUI 400 comprises a battery indicator 407 configured to indicate the relative charge left in the battery. The GUI 400 also comprises a signal indicator 409, informing convey information to a user indicating the data-communication connection strength of the communication interface 204, with any device to which it is connected. The GUI 400 also comprises a title indicator portion 405 configured to show a title of a particular recipe that has been chosen by a user of the device, the recipe comprising specific relative amounts of liquids or powders comprised in the cartridges. The GUI 400 also comprises a descriptor indicator portion 406 configured to show a description of a particular recipe that has been chosen by a user of the device. The GUI 400 also comprises graphical control elements 403 and 404, allowing a user to control operation of the device 100. Non-limiting examples of graphical control elements include graphical control element 403 configured to allow a user to cycle through recipes that are stored in memory 216, and graphical control element 404 configured to allow a user to request more recipes from server 1310 or device 1301.

The GUI 400 also comprises cartridge information indicators 408₁₋₅, showing the contents of the cartridges, e.g. tetrahydrocannabinol (THC), tetrahydrocannabinolic acid (THCA), cannabidiol (CBD), delta-9-tetrahydrocannabivarin (THCV) and limonene (LMN), as well as the relative composition indictors 408₁₋₅ showing the relative amounts of the cartridges' contents that are to be dispensed through spout 401 by the device using the system shown in FIG. 2 and described above. In some embodiments, the GUI 400 is a touch screen, and the relative amounts can be set by a user by setting the relative composition indictors 408₁₋₅ manually. In some embodiments, the GUI 400 comprises cartridge volume indicators 410₁₋₅ configured to convey information relating to the amount of liquid or powder remaining in each cartridge. In some examples, the cartridge volume indicators 410₁₋₅ are configure to alert a user when the amount of liquid or powder in a given cartridge falls below a threshold. In some embodiments, the processor is configured to generate a low volume alert signal that can optionally be communicated to the user. As represented in FIG. 4, cartridge volume indicator 410₁ is blinking/flashing because its associated cartridge (THC) is almost empty.

As herein defined, a composition or formulation is a recipe including one or more cannabinoid-containing additives, which is suitable for adding to a beverage, or a beverage containing one or more cannabinoid-containing additives.

In operation, when a user selects a particular composition, the processor 201 receives the formulation instructions form the GUI 203 or the memory 216 and/or any other device such as, but not limited to, devices 1306, 1307, 1308, 1301 and 1310 shown in FIG. 13, via the communication interface 204. Once received, the processor determines the required amounts of the contents of each cartridge required for the particular composition. For example, if a particular composition requires 40 mg of THC, 30 mg of CBD, 20 mg of limonene, 40 mg of citrus flavour, the processor 201 determines the contents of the cartridges 208₁₋₅, by querying memory 216 for relevant cartridge information. Upon receipt of the cartridge information, the processor can determine whether the requested formulation can be achieved, based on the nature and the amount of contents of the cartridges 208₁₋₅. For example, if cartridge 208₁ contains limonene, cartridge 208₃ contains THC, and cartridge 208₄ contains CBD, it will be possible to produce the required formulation, provided that each cartridge has sufficient amounts of liquid or powder, and that the concentrations of THC, CBD and limonene are sufficient to produce the desired formulation, given the remaining amounts of liquid or powder.

Once a determination is made that the device 200 can produce the desired additive, the processor 201 sends instructions to the valve controller 202 to meter out the appropriate amount of material from each cartridge 208ₓ. Once the amounts are metered out, the metered out material is dispensed through the spout 206 into a receptacle 101, which could be empty or pre-filled with a carrier beverage into which the formulation is to be added.

In some examples, a large cartridge (or refillable tank) 205 is also included in the device 205. The large cartridge (or refillable tank) 205 can comprise a carrier beverage. Examples of carrier beverages include, but are not limited to, flavoured water (either carbonated or not), fruit juices and brewed beverages such as coffee or tea. The large cartridge (or refillable tank) 205 is in fluid communication with a metering valve 206, which metering valve is controllable by the valve controller 202. Accordingly, the valve controller is operable to control the amount of carrier beverage that is dispensed through by the device 200 through spout 206.

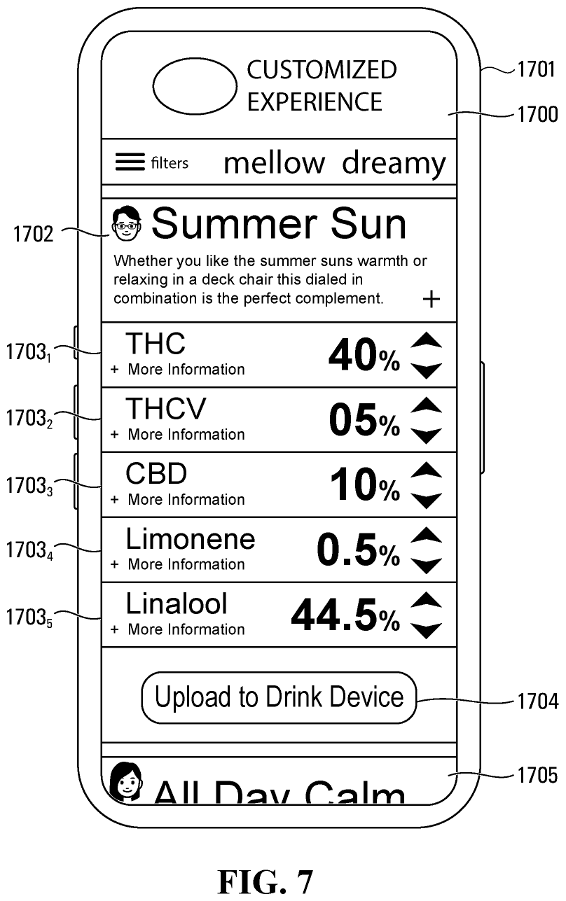

FIG. 13 shows a high-level network diagram of an example data-communication system configured to be used with example devices for preparing cannabinoid-containing beverages. In some embodiments, devices 1302, 1303, 1304 for preparing cannabinoid-containing beverages are equipped to be controlled by a local communication device 1301. In some embodiments, the local communication device is a mobile communication device 1301, such as a smartphone or tablet. As shown in FIG. 7, in some embodiments, the mobile device 1301, 1701 is configured to implement a Graphical User Interface (GUI) 1700 allowing a user to control devices 1302, 1303, 1304. In some embodiments, the GUI is implemented by a mobile application (or “app”). In some embodiments, the GUI 1700 comprises a title and descriptor area 1702, in which the title of a formulation or recipe is shown, and a description of the formulation or recipe is shown. In some embodiments, the description of the recipe comprises information conveying a feeling or experience which may be achieved by consumption of the formulation. In some embodiments, the description of the recipe comprise information conveying an appropriate activity during which the formulation can be consumed. In some embodiments, the description of the recipe comprise information conveying an appropriate setting in which the formulation can be consumed. The GUI 1700 also comprises graphical control elements 1703₁₋₅ which serve to convey information about a formulation to a user, and to allow a user to change the compositions of formulations, by interacting with the graphical control elements to dial the amount of any cannabinoid-containing substance (or other additive or flavoring) either up or down, by pressing the appropriate graphical control element arrows. In some embodiments, the graphical control elements 1703₁₋₅ also allow a user to seek more information (e.g. be pressing the “+More Information” graphical control element) about a particular cannabinoid-containing substance (or other additive or flavoring).

In some embodiments, the GUI 1700 comprises a graphical control element 1704 allowing a user to instruct the mobile device 1301, 1701 to upload a set formulation to device 1302, 1303. In some examples, the set formulation is a formulation or recipe that has been presented to the user by the mobile application. In other examples, the set formulation is a formulation or recipe that has been at least partially set by the user interacting with the graphical control elements.

In some embodiments, the device 1302, 1303, 1304 can be enabled by using the authentication mechanism of the mobile device 1301. For example, the device 1302, 1303, 1304 can be disabled until such time that the mobile application running on the mobile device 1301 issues an activation signal to the device 1302, 1303, 1304. The activation signal in turn can be issued by the mobile application accessing the authentication mechanism of the mobile device (e.g. fingerprint recognition mechanism, PIN code entry and/or facial recognition mechanism) in order to authenticate the user. This method can also be used to ensure child-resistance.

In some embodiments, the title and descriptor area 1705 of the next formulation accessible by the mobile application can be seen at the bottom of the GUI 1700, and the GUI is configured to allow the user to scroll up and/or down to browse through various formulations. In some embodiments, the mobile application is configured to collect formulation browsing information indicative of the formulations browsed by the user.

In some embodiments, and as described above, devices 1302, 1303, 1304 for preparing cannabinoid-containing beverages and/or mobile device 1301 are configured to communication with other devices 1306 , 1307, 1308 over the internet in order to share transmit and receive formulations, cartridge information and other information related to the use of devices 1302, 1303, 1304. In some embodiments, devices 1302, 1303, 1304 are also configured to communicate with server 1310, which may be a server relating to the manufacturer, distributor and/or vendor of the cartridges containing the cannabinoid-containing substances or other additives and/or devices 1302, 1303, 1304. In some embodiments, use information generated by devices 1302, 1303, 1304 and/or mobile device 1301 can be sent to server 1310. Use information can include, but is not limited to, cartridge information, cartridge content information, formulation browsing information, the number of times a device 1302, 1303, 1304 has been used to dispense a formulation, the compositions of the formulations dispensed by a device 1302, 1303, 1304, the geographic location of a device 1302, 1303, 1304, the registered owner/user of a device. In some embodiments, the server 1310 can store user data in secured database 1312, and anonymized use and/or user data, and other related data in database 1311. Examples of anonymized user data are geographical location of devices, the time of day that certain formulations have been dispensed, etc. In some embodiments, the anonymized data can be run through various analytics engines 1309 in order to determine user preference information for certain cannabinoid-containing substances, cartridges, devices 1302, 1303, 1304 and/or compositions. In some embodiments, the user preference information can be used to provide a user with recommendations comprising recommended formulations, cannabinoid-containing substances, flavouring additives and/or other additives. In some embodiments, the recommendations can be provided to the user by way of devices 1302, 1303. In some embodiments, the recommendations can be provided to the user by way of the mobile application running on mobile device 1301.

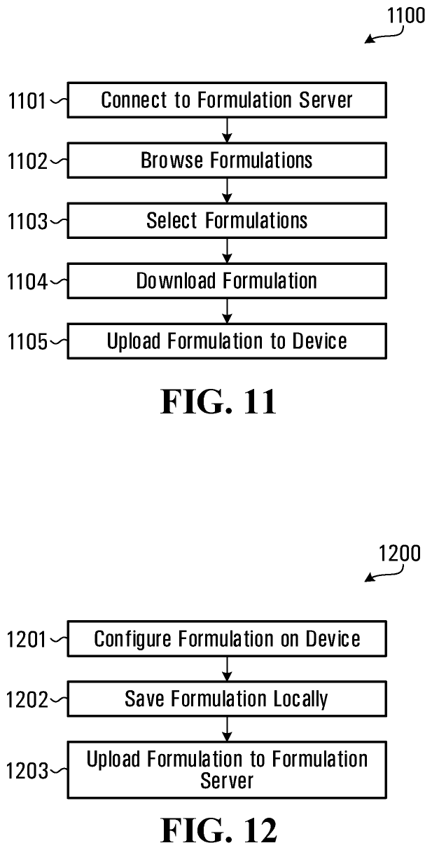

FIG. 11 shows a flowchart representing an example method of downloading a formulation recipe for use with an example device for preparing cannabinoid-containing beverages. At step 1101, device 1302, 1303, 1304 or mobile device 1301 connects to a formulation server configured to provide data indicative of various formulations. In some embodiments, the formulation server is server 1310. At step 1102, a user can browse formulations using the GUI of device 1302 or the GUI of mobile application 1302. At step 1103, the user can select a formulation using one of the graphical control elements of the GUI of device 1302 or the GUI of mobile application 1302. After selecting the formulation, the formulation data can be downloaded to the device 1302, 1303, 1304 or mobile device 1301 at step 1104. The formulation data comprises any data required for the device 1302, 1303, 1304 to dispense the formulation. Optionally, if the formulation data is downloaded to mobile device 1301, the formulation data can then be uploaded to device 1302, 1303, 1304 at step 1105.

FIG. 12 shows a flowchart representing an example method of uploading a formulation recipe for use with an example device for preparing cannabinoid-containing beverages. At step 1201, a formulation can be configured by a user on device 1302, or can be configured on the mobile device 1301 for use on device 1302, 1303, 1304. At step 1202, the formulation can be saved locally on device 1302, 1303, 1304 or mobile device 1301. At step 1203, the configured formulation can be uploaded from device 1302, 1303, 1304 or mobile device 1301 to a formulation server. In some embodiments, formulation server is server 1310.

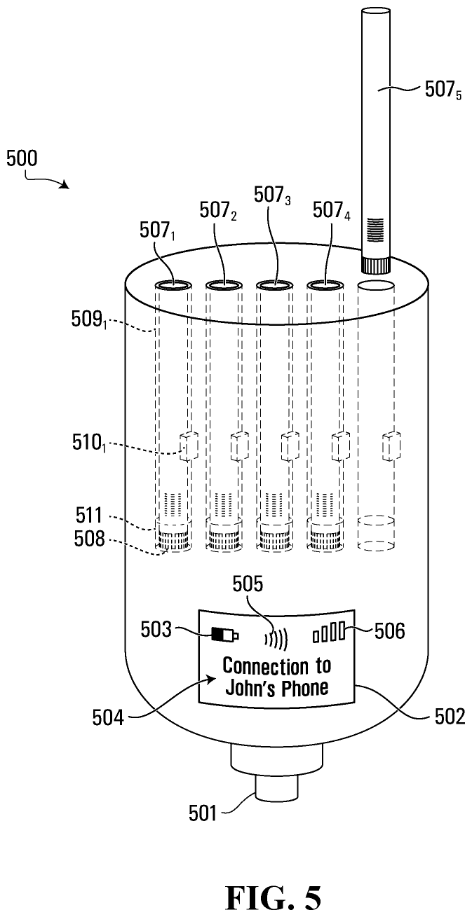

FIG. 5 shows a diagram of an example hand-held device 500 for preparing liquid formulations of cannabinoid-containing beverages. The device is configured to dispense cannabinoid-containing substances to be added to existing beverages.

The device 500 comprises a plurality of slots 509₁₋₅ configured to receive a plurality of cartridges 507₁₋₅. In the embodiment shown in FIG. 5, the device 500 comprises five slots 507ₓ, though in other embodiments, the device 500 can include any number of slots 507ₓ, for example ten slots or twenty slots.

With reference to slot 507₁, in some embodiments, each slot 507ₓ comprises a device interface 511 for connecting to a corresponding cartridge interface 508 forming part of cartridge 507₁. Interfaces 511 and 508 allow for liquid or powder to flow from the cartridge 507₁, into the device 500. In some embodiments, interfaces 511 and 508 allow for securing cartridge 507₁ to the device 500. In some embodiments, interfaces 511 and 508 also allow for electronic, chemical, or electromechanically communication between functional elements of cartridge 507₁ and the device 500. Each slot 509 also comprises a cartridge tag reader 510 configured to read a cartridge tag included on (or in) each cartridge 507ₓ. The device also includes a spout 501 in controlled fluid communication with the device interface of each slot 509.

In some examples, each cartridge 507ₓ comprises a cartridge tag. The cartridge tag is configured to convey information relating to the cartridge 507ₓ to device 500. Examples of information relating to the cartridge include, but are not limited to, the contents of the cartridge 507ₓ, the manufacturer, distributor and/or vendor of the cartridge 507ₓ, information relating to the shelf life of the contents of the cartridge 507ₓ.

In some examples, the cartridge tag is a barcode and each slot 509 includes a barcode reader 510 configured to read the barcode when a cartridge 507ₓ is inserted in the device slot 509, or thereafter. Other suitable cartridge tags include, but are not limited to, Quick Response (QR™) codes, or other suitable matrix barcodes, Radio Frequency Identification (RFID) tags, or any other suitable tag able to convey the aforementioned information relating to the cartridge 507ₓ. Similarly, other suitable cartridge tag readers 510 include, but are not limited to, Quick Response (QR™) code readers, or other suitable matrix barcode readers, Radio Frequency Identification (RFID) tag readers, or any other suitable tag readers able to read tags configured to convey the aforementioned information relating to the cartridge 507ₓ. The device 500 also comprises a GUI 504, including a battery indicator 503, a cellular communication signal strength indicator 506, a WiFi signal strength indicator 505 and a display area 504 for displaying a description of a particular recipe that has been chosen by a user of the device, graphical control elements allowing a user to control operation of the device. Non-limiting examples of graphical control elements include graphical control element configured to allow a user to cycle through recipes that are stored in memory, and graphical control element configured to allow a user to request more recipes from server 1310 or device 1301.

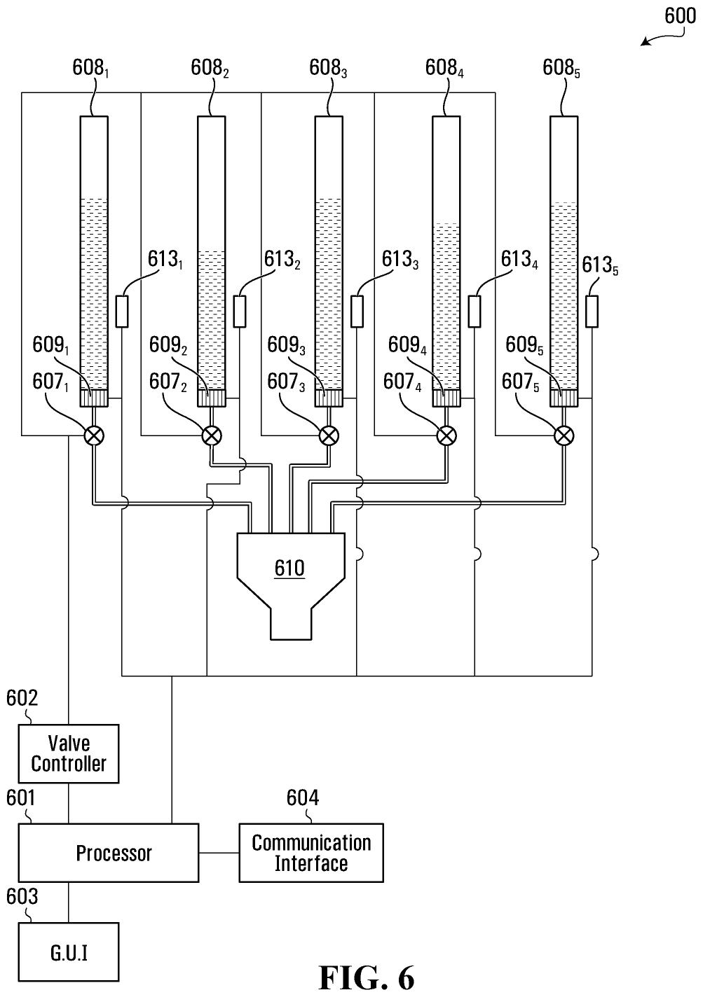

Aspects of the operation of device 500 will now be described with particular reference to FIG. 6, which shows a schematic diagram of an example device for preparing and dispensing cannabinoid-containing beverages. The device 600 comprises a plurality of couplings 609₁₋₅ configured to allow free fluid communication of powders or liquids contained in cartridges 608₁₋₅ into respective metering valves 607₁₋₅. In some embodiments, each coupling 609ₓ consists of a device interface 511 operably engaged with a cartridge interface 508 to allow substantially unobstructed flow of powders or liquids contained in cartridges 608₁₋₅ into respective metering valves 607₁₋₅.

Device 600 also comprises a processor 601 operably connected to a valve controller 602, a communication interface 604, a memory and a Graphical User Interface (GUI) 603. The valve controller 602 is operably connected to metering valves 607₁₋₅. In some embodiments, two or more elements of the processor 601, the valve controller 602 and the communication interface 604 may be implemented by devices that are physically distinct from one another and may be connected to one another via a bus (e.g., one or more electrical conductors or any other suitable bus) or via a communication link which may be wired. In other embodiments, two or more elements of the processor 601, the valve controller 602 and the communication interface 604 may be implemented by a single integrated device. Moreover, in some embodiments, two or more elements of the processor 601, the valve controller 602, the metering valves 607₁₋₅, the cartridge tag readers 613ₓ and the communication interface 604 may be connected to one another via a bus (e.g., one or more electrical conductors or any other suitable bus) or via a communication link which may be wired.

In some embodiments, the device 600 comprises multiple metering valves 607₁₋₅ operable to provide electronically controlled accurate and stable flow rates into spout chamber 610. The flow rate and flow duration of each metering valve 607ₓ is controlled by valve controller 602, using instructions received from processor 601. In other embodiments, metering valves 607₁₋₅ can be replaced by any suitable device configured to precisely meter out (or otherwise dispense with substantial precision) the contents of the cartridges 608₁₋₅ into the spout chamber 610 under control of the valve controller 602, or other equivalent controller.

In some embodiments, the device 600 comprises cartridge tag readers 613₁₋₅ for each cartridge 608₁₋₅ respectively. In some examples, each cartridge 608ₓ comprises a cartridge tag (not shown in FIG. 6). As described above, the cartridge tag is configured to convey information relating to the cartridge 608ₓ to device 600. Examples of information relating to the cartridge include, but are not limited to, the contents of the cartridge 608ₓ, the manufacturer, distributor and/or vendor of the cartridge 608ₓ, information relating to the shelf life of the contents of the cartridge 608ₓ. Once read by a cartridge tag reader 613ₓ, the information relating to the cartridge can be stored in the memory for later retrieval by, for example, the processor 601.

In some examples, each cartridge tag reader 613ₓ includes a barcode reader configured to read a barcode contained on a cartridge 608ₓ when the cartridge 608ₓ is inserted into the device. In some embodiments, the barcode required to comply with regulatory requirements relating to cannabis-based product packaging is the barcode used to identify the cartridge. Other suitable cartridge tags include, but are not limited to, Quick Response (QR™) codes, or other suitable matrix barcodes, Radio Frequency Identification (RFID) tags, or any other suitable tag able to convey the aforementioned information relating to the cartridge 608ₓ. Similarly, other suitable cartridge tag readers 613 include, but are not limited to, Quick Response (QR™) code readers, or other suitable matrix barcode readers, Radio Frequency Identification (RFID) tag readers, or any other suitable tag readers able to read tags configured to convey the aforementioned information relating to the cartridge 608ₓ.

In some embodiments, once the cartridge tag reader 613 reads the information relating to the cartridge 608ₓ from the cartridge tag, the information is sent to processor 601. Processor 601 is configured to use the information relating to the cartridge in a number of ways, as will be described in more detail below.

In some embodiments, the spout chamber 610 is configured to mix any powder, liquid and gas dispensed from the metering valves 607ₓ before dispensing the resulting mixture into, for example, a beverage receptacle. In other embodiments, the spout chamber 610 is configured to directly dispense any substance dispensed from the metering valves 607ₓ into a beverage receptacle. The aforementioned mixing and/or dispensing steps can be controlled by processor 601.

Processor 601 is configured to generally control operations of the device based on information received from the communication interface 604 and/or the GUI 502. Accordingly, the processor 601 is configured to receive and send information from and to the communication interface 604 and the GUI 502. Moreover, processor 601 is configured to receive information related to the cartridges received form cartridge tag readers 613₁₋₅, as well as information relating to the remaining volume of liquid or powder remaining in the cartridges 608₁₋₅ from the volume sensor 301 in each cartridge 608₁₋₅, by way of couplings 609₁₋₅. Processor 601 is configured to communicate with each metering valve 607₁₋₅ in order to precisely control the amount of liquid or powder that is dispensed from each metering valve 607₁₋₅ by way of valve controller 602.

In some examples, the device also comprises a communication interface 604 for communicating with other devices. As shown in FIG. 13, in some examples, the communication interface 604 of the device 1303 may interact with other devices over a communication link, which may be wireless, wired, or partly wireless and partly wired (e.g., Bluetooth or other short-range or near-field wireless connection, WiFi or other wireless LAN, WiMAX or other wireless WAN, cellular, Universal Serial Bus (USB), etc.). In some examples, the other devices can include a smartphone 1301, 1306, 1307 or other wireless phone; a tablet computer 1308; a head-mounted display, smartwatch or other device; or any other communication device carried, worn or otherwise associated with a user. In some examples, the other device can include a server 1310 or other computing entity (e.g., implementing a website) associated with a manufacturer, distributor or vendor of the cartridges

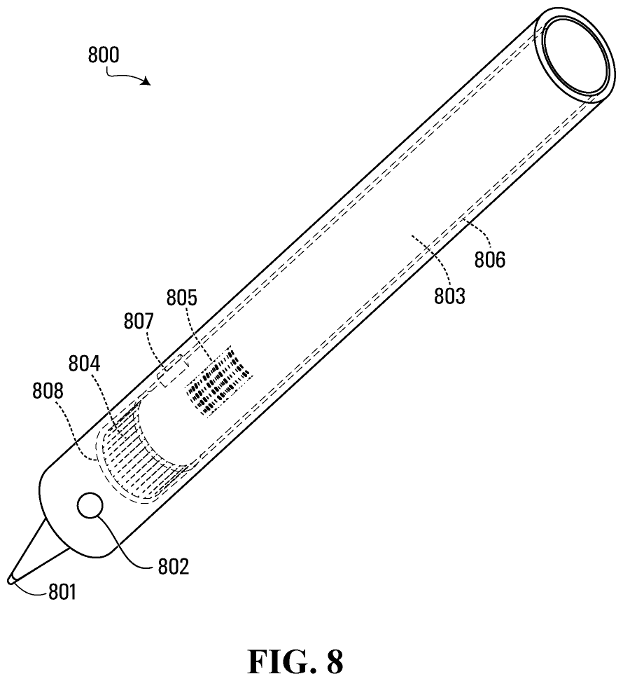

FIG. 8 shows a diagram of an example device 800 for dispensing a liquid formulation for making a cannabinoid-containing beverages. The device 800 is configured to dispense cannabinoid-containing substances to be added to existing beverages found in a beverage receptacle. Device 800 is a hand-held device for dispensing a substance from a single cartridge 803. The device 800 comprises a single slot 806 configured to receive a single cartridge 803.

In some embodiments, the slot 806 comprises a device interface 808 for connecting to a corresponding cartridge interface 804 forming part of cartridge 803. Interfaces 808 and 804 allow for liquid or powder to flow from the cartridge 803, into the device 800. In some embodiments, interfaces 808 and 804 allow for securing cartridge 803 to the device 800. In some embodiments, interfaces 808 and 804 also allow for electronic, chemical, or electromechanically communication between functional elements of cartridge 803 and the device 800. The slot 806 also comprises a cartridge tag reader 807 configured to read a cartridge tag 805 included on (or in) the cartridge 803. The device 800 also includes a spout 801 in controlled fluid communication with the device interface 808.

In some examples, each cartridge 803 comprises a cartridge tag 805. The cartridge tag 805 is configured to convey information relating to the cartridge 806 to device 800. Examples of information relating to the cartridge include, but are not limited to, the contents of the cartridge 806, the manufacturer, distributor and/or vendor of the cartridge 806, information relating to the shelf life of the contents of the cartridge 806.

In some examples, the cartridge tag 805 is a barcode and each slot 806 includes a barcode reader 807 configured to read the barcode when a cartridge 803 is inserted in the device slot 806, or thereafter.

In some embodiments, the device 800 comprises an actuator 802 configured to cause the device 800 to dispense a dose of the substance contained in the cartridge 803. Similarly to the devices of FIGS. 1 and 5, the device 800 is configured to communicate with a communication device 1301. In some embodiments, the device 800 and the communication device 1301 are configured to allow the size of the dose to be set by the communication device 1301. In some embodiments, the size of the dose can be set by a mobile application running on the communication device 1301. The mobile application can set the size of the dose based on a predetermined formulation (e.g. downloaded from the server 1310, or received from one or device 1306, 1307, 1308), or can be set by a user using the mobile application.

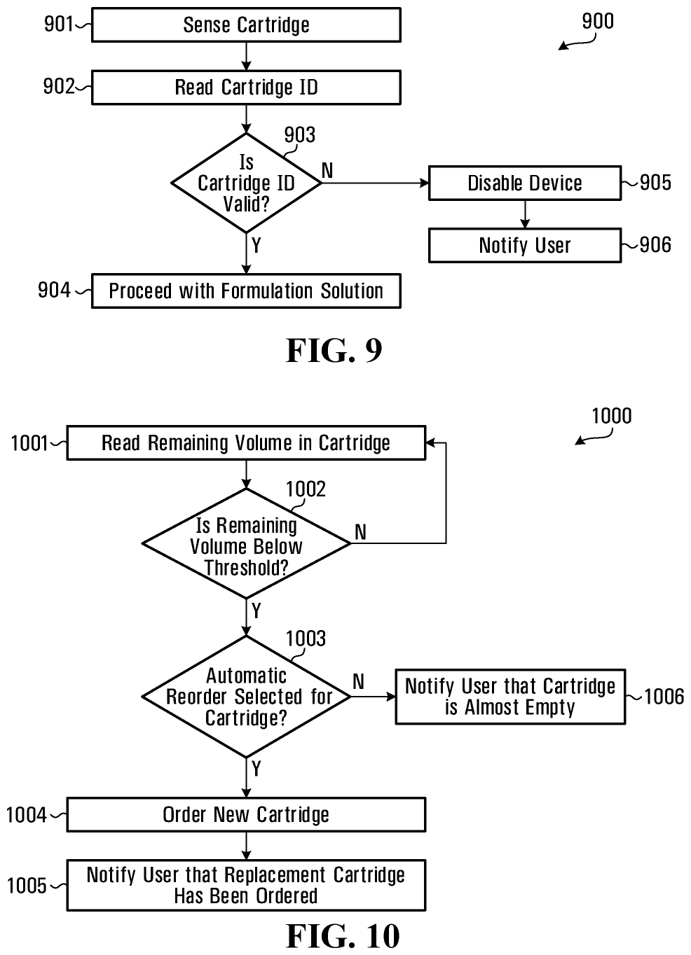

With particular reference to FIG. 9, a method of validating a cartridge for use in the device is shown. While this method will be explained with reference of the device of FIG. 1, it will be understood that the method can also be carried out on the device of FIGS. 5 and 8. At step 901, when a user inserts a cartridge 104ₓ in an interface of the device 100, the device senses the presence of the cartridge 104ₓ. Sensing (or detecting) the presence of the cartridge 104ₓ can be accomplished any number of ways including, but not limited to, detecting the presence of the cartridge interface 105ₓ by any suitable electromechanical, electronic or optoelectronic means. Once the device 100 detects the presence of a cartridge, the device reads the cartridge tag in order to derive cartridge information therefrom. In some examples, the device 100 comprises a barcode reader for reading a barcode 106 on each cartridge 104ₓ. In some examples, the device comprises an RFID reader for reading the RFID on each cartridge 104ₓ.

Once the cartridge information is read by the device 100, a determination is made as to whether or not the cartridge information of the cartridge is valid at step 903. In some examples, the cartridge information includes information relating to the contents of the cartridge 104ₓ and/or the identity of the manufacturer, distributor and/or vendor of the cartridge 104ₓ. If a determination is made that the cartridge information is valid, then the device allows a user to prepare beverages with the contents of the cartridge. If, on the other hand, a determination is made that the cartridge information is invalid, then the device is configured to prevent a user from preparing a beverage with the contents of the cartridge. In some examples, such functionality is provided by enabling or disabling the relevant controllable valve 207, which is in fluid communication with the cartridge 104ₓ that is being validated. Examples of valid cartridge information include, but are not limited to, information indicating that a cartridge contains a particular product in a particular concentration (e.g. water soluble THC of a particular concentration), or that the cartridge 104ₓ was made by a given manufacturer, distributed by a given distributor, and/or sold by a given vendor. Examples of invalid cartridge information include, but are not limited to, information indicating that a cartridge contains a particular product in a particular concentration that is not allowed by one of the settings of the device 100 (e.g. water soluble THC of a particular concentration that is above a certain threshold), or if the cartridge was not made by a particular manufacturer, not distributed by a particular distributor, and/or not sold by a particular vendor.

At step 905, after a determination is made that the cartridge is not valid (for any one or more of the reasons set out above, or any other reason), the device disables the interface and/or valve associated with the interface in such a way as to prevent the user from dispensing the contents of the cartridge in a beverage using the device 100. Then at step, 906, the user can be notified of the fact that the device 100 has detected an invalid cartridge 104ₓ. Alternatively, at step 904, after a determination is made that the cartridge is valid (for any one or more of the reasons set out above, or any other reason), then the device does not disables the interface and/or valve associated with the interface in such a way as to allow the user to dispense the contents of the cartridge in a beverage using the device 100.

FIG. 10 shows a flowchart representing an example method of automatically reordering a cartridge for use with an example device for preparing cannabinoid-containing beverages. While this method will be explained with reference of the device of FIG. 1, it will be understood that the method can also be carried out on the device of FIGS. 5 and 8. At step 1001, the device 100 reads the remaining volume of substance in a cartridge 104ₓ, in accordance with the above. At step 1002, a determination is made as to whether the remaining volume of substance is below a certain threshold. If the remaining volume is below a certain threshold, the device 100 determines whether the device is set to automatically reorder cartridges at step 1003. In some examples, the user will have enabled this feature in order to avoid running out of substances for use in creating cannabinoid-containing beverages. If the automatic reorder feature is not enabled, the device can notify the user that the cartridge is almost empty at step 1006. If the automatic reorder feature is enabled, the device can use the communication interface 604 to contact a reorder server to order a new cartridge, at step 1004. In some embodiments, reorder server is server 1310. Finally, at step 1005, the device can notify the user that a replacement cartridge has been ordered at step 1005. In some embodiments, the device can notify the user by way of the GUI 400.

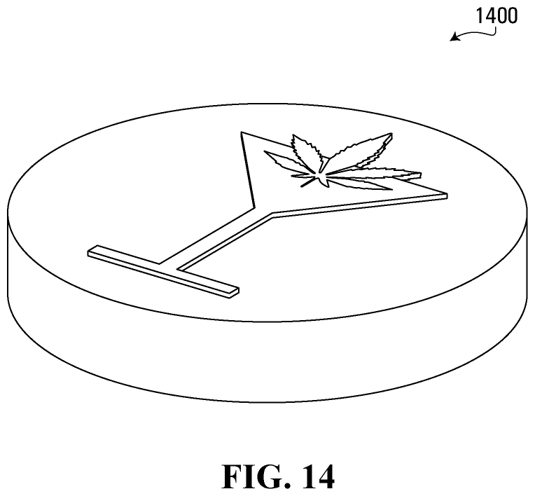

With reference to another aspect, FIG. 14 shows an example effervescent tablet for creating a cannabinoid-containing beverage. In some embodiments, the formulation is in the form of a tablet that effervesces in water or an aqueous solution. The effervescence is a result of CO₂ liberation from the reaction of an acid and a base contained in the tablet with water. Effervescence helps to evenly and rapidly disperse the cannabinoid-containing substance as fine particles in solution, which is particularly important for compounds that are insoluble or sparingly soluble in water, such as cannabinoids, terpenes and terpenoids. Effervescent formulations of poorly water-soluble compounds have been shown to provide better bioavailability and absorption of such compounds as effervescence helps to create finer particles that are less likely to precipitate in the gastrointestinal tract. Components of effervescent formulations may include acids and bases, lubricants, surfactants, binders, sweeteners, flavouring agents, colouring agents, or any combination thereof.

Acids that may be used in effervescent tablets may include, but are not limited to, citric acid, tartaric acid, ascorbic acid, fumaric acid, acetylsalicylic acid, malic acid, acid anhydrides, and acid salts. Bases that serve as a source of CO₂ in effervescent tablets may include, but are not limited to, sodium bicarbonate, sodium carbonate, potassium carbonate, potassium carbonate, calcium carbonate, and amino acid-alkali metal carbonate derivatives such as sodium glycine carbonate. In some embodiments, to avoid a premature effervescent reaction, the entire process of tablet production and storage is be performed at low moisture.

In some embodiments, an intrinsic lubricant may be added to the formulation to ensure that all components of the tablet are well-mixed prior to tableting and are free-flowing as a solid mixture. Lubricants also ensure that the tablet does not stick to any of the machining components upon compression.

In some embodiments, a lubricant with both hydrophobic and hydrophilic properties is used. Surfactants such as sodium lauryl sulfate and magnesium lauryl sulfate can also be used as lubricants.

In some embodiments, surfactants are added to the effervescent tablets to increase the dissolution rate of compounds in the tablet. Moreover, in some embodiments, an anti-foaming agent is used, such as poly-dimethylsiloxane, to reduce the tendency of compounds to stick to the wall of the formulation container above the water level.

In some embodiments, binders are used to hold the tablet together (e.g., maltitol). In some embodiments, sweeteners such as sucrose and sorbitol are used in the tablet.

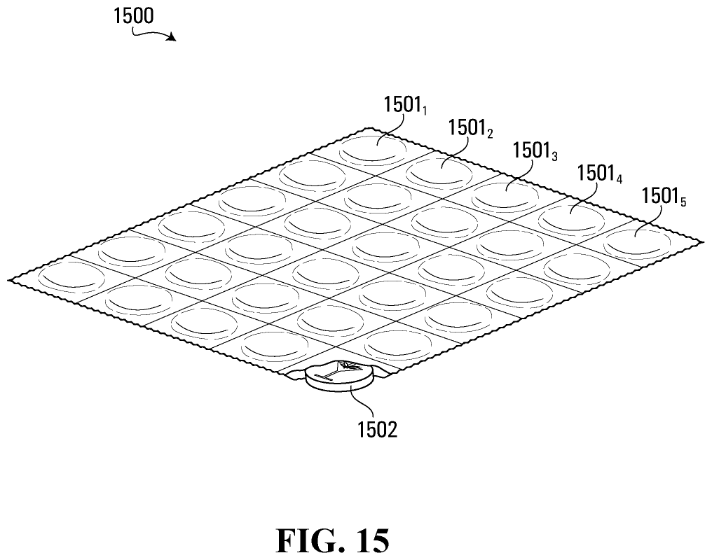

With particular reference to FIG. 15, an example consumer product for the safe distribution and sale of a plurality of example effervescent tablets will now be described. The consumer product 1500 comprises a connected array of individually-wrapped effervescent tablets 1502. Each tablet 1502 contains a liquid-soluble or partially liquid-soluble cannabinoid-containing substance. In some embodiments, each effervescent tablet 1502 contains a different liquid-soluble or partially liquid-soluble cannabinoid-containing substance. In some embodiments, the effervescent tablets in a row 1501ₓ of the plurality of rows 1501₁₋₅ of the array contain the same liquid-soluble or partially liquid-soluble cannabinoid-containing substance. In some embodiments, each column of the array contains effervescent tablets 1501 containing different liquid-soluble or partially liquid-soluble cannabinoid-containing substances. In some embodiments, at least one row 1501ₓ of the plurality of rows 1501₁₋₅ of the array contains effervescent tablets containing a tertrahydrocannabinol (THC)-based substance. In some embodiments, at least one row 1501ₓ of the plurality of rows 1501₁₋₅ of the array contains effervescent tablets containing a substance configured to act as an antidote to a tertrahydrocannabinol (THC)-based substance. In some embodiments, at least one row 1501ₓ of the plurality of rows 1501₁₋₅ of the array contains effervescent tablets containing a cannabidiol (CBD)-based substance.

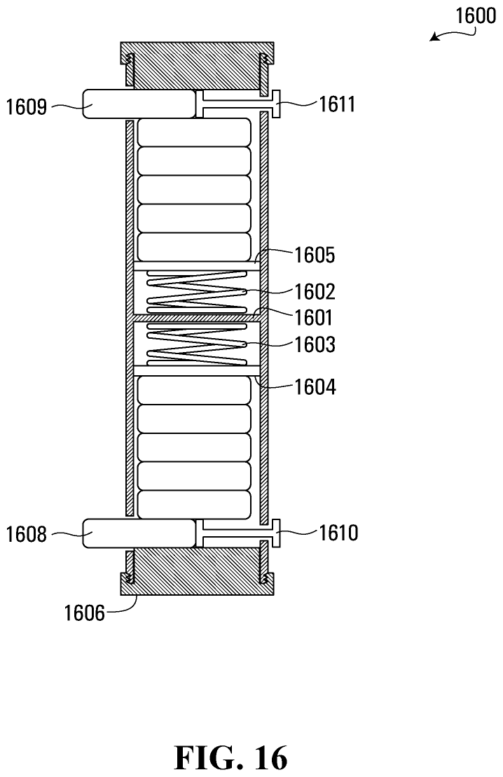

FIG. 16 shows and example of consumer product packaging for the safe distribution and sale of a plurality of example effervescent tablets. The device 1600 is configured to dispense effervescent tablets comprising at least one liquid-soluble or partially liquid-soluble cannabinoid-containing substance. The device 1600 comprises two chambers, each configured to hold a plurality of effervescent tablets. Each chamber comprising a dispensing end have an opening configured to allow an effervescent tablet to pass therethrough. In the example of FIG. 16, each chamber can be filled with tablets by opening the chamber via a screw top 1606. Each chamber also comprises mechanical biasing means for biasing the effervescent tablets towards the dispensing end. In some embodiments, the mechanical biasing means includes a platform 1605, 1604 and spring 1602, 1603. The device 1600 also comprises a dispensing actuator 1610, 1611 configured to allow a user to move one effervescent tablet at least partially through the opening.

The skilled reader will readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The embodiments are also intended to cover computers programmed to perform said steps of the above-described methods.

It should be appreciated by those skilled in the art that any block diagrams herein represent conceptual views of illustrative circuitry embodying the principles disclosed herein. Similarly, it will be appreciated that any flow charts and scripts and the like represent various processes which may be substantially represented in computer readable medium and so executed by a computer or processor, whether or not such computer or processor is explicitly shown.

Although various embodiments and examples have been presented, this was for purposes of description, but should not be limiting. Various modifications and enhancements will become apparent to those of ordinary skill in the art.

Certain additional elements that may be needed for operation of some embodiments have not been described or illustrated as they are assumed to be within the purview of those of ordinary skill in the art. Moreover, certain embodiments may be free of, may lack and/or may function without any element that is not specifically disclosed herein.

Any feature of any embodiment discussed herein may be combined with any feature of any other embodiment discussed herein in some examples of implementation.

1. An apparatus for making cannabinoid-containing beverages, the apparatus comprising:
at least one interface configured to receive a cartridge containing a cannabinoid-containing substance;
at least one valve, in fluid communication with the at least one interface, configured to dispense an amount of the substance from the cartridge;
a valve controller configured to control the valve in order to vary the amount of the substance dispensed by the valve;
a cartridge tag reader configured to read a cartridge tag associated with a cartridge, the cartridge tag being arranged to convey cartridge information; and
a processor configured to disable the apparatus based on the cartridge information.

2-7. (canceled)

8. The apparatus of claim 1, wherein the apparatus further comprises:
a communication interface configured to receive instructions from a communication device;
wherein the processor is further configured to receive the instructions from the communication interface, and to control the operation of the valve controller such that the amount of the substance dispensed by the valve can be set by the communication device.

9. The apparatus of claim 8, wherein the communication device is a mobile device, and wherein the instructions are generated on the mobile device using a mobile application relating to the apparatus.

10-11. (canceled)

12. The apparatus of claim 1, wherein the cartridge information comprises at least one of: information identifying the substance contained in the cartridge, information identifying the manufacturer of the cartridge, information identifying the distributor of the cartridge and information identifying the vendor of the cartridge.

13. (canceled)

14. The apparatus of claim 12, wherein the processor is configured to disable the apparatus unless the information identifying the manufacturer of the cartridge comprises information indicating that the cartridge was manufactured by a pre-determined manufacturer.

15. (canceled)

16. The apparatus of claim 1, wherein the cartridge tag comprises one of a linear barcode and a 2-D barcode, and wherein the barcode is required for regulatory compliance.

17. The apparatus of claim 8, wherein the processor and the communication interface are configured to provide the cartridge information to the communication device.

18-20. (canceled)

21. The apparatus of claim 1, wherein the apparatus comprises:
a plurality of interfaces, each configured to receive one of a plurality of cartridges containing a cannabinoid-containing substance; and
a plurality of valves, each in fluid communication with a respective interface, and configured to dispense an amount of the substance from a respective cartridge.

22-26. (canceled)

27. The apparatus of claim 21, wherein the apparatus further comprises:
a communication interface configured to receive instructions from a communication device; and
the processor is further configured to control the operation of the valve controller such that the amounts of the substances dispensed by the plurality of valves can be set by the instructions received from the communication device.

28-29. (canceled)

30. The apparatus of claim 21, wherein the apparatus further comprises:
a Graphical User Interface (GUI) configured to implement a plurality of graphical control elements, the graphical control elements being configured to allow a user of the apparatus to select the amounts of the substances dispensed by the plurality of valves.

31. The apparatus of claim 30, wherein the graphical control elements are further configured to allow a user to download, over a data-communication network, instructions for controlling the operation of the valve controller to set given amounts of the substances dispensed by the plurality of valves.

32. The apparatus of claim 21, wherein the apparatus comprises a plurality of cartridge tag readers, each configured to read a cartridge tag associated with a cartridge of the plurality of cartridges, each cartridge tag being arranged to convey cartridge information relating to a respective cartridge.

33. (canceled)

34. The apparatus of claim 32, wherein the processor is further configured to disable the apparatus based on the cartridge information of at least one of the cartridge tags.

35-41. (canceled)

42. A computer-implemented method of controlling an apparatus for making cannabinoid-containing beverages, the apparatus comprising at least one interface configured to receive a cartridge containing a cannabinoid-containing substance and a cartridge tag, and at least one cartridge tag reader configured to read the cartridge tag, the cartridge tag being configured to convey cartridge information relating to the cartridge, the method comprising the steps of:
reading the cartridge information from the cartridge tag; and
disabling the apparatus if the cartridge information does not correspond to predetermined cartridge information.

43. The computer-implemented method of claim 42, wherein the cartridge information comprises at least one of: information identifying the substance contained in the cartridge, information identifying the manufacturer of the cartridge, information identifying the distributor of the cartridge and information identifying the vendor of the cartridge.

44. The computer-implemented method of claim 43, wherein the cartridge information and the predetermined cartridge information are related to information identifying the manufacturer of the cartridge.

45. The computer-implemented method of claim 42, wherein the apparatus further comprises a Graphical User Interface (GUI) and the method further comprises:
generating a notification on the GUI indicating that the apparatus has been disabled.

46-47. (canceled)

48. A computer-implemented method of controlling the apparatus of claim 27, the method comprising the steps of:
connecting to a formulation server configured to provide information indicative of a plurality of formulations of different combinations of cannabinoid-containing substances;
selecting a formulation from among the plurality of formulations;
receiving the information indicative of the selected formulation at the communication device;
receiving the information indicative of the selected formulation at the apparatus;
configuring the valve controller in accordance with the selected formulation.

49. A cartridge for use with an apparatus of claim 1, the cartridge comprising:
an amount of a cannabinoid-containing substance; and
a cartridge tag configured to convey cartridge information, which relates to the cartridge and based upon which the apparatus may be disabled.

50. The cartridge of claim 49, wherein the cartridge information comprises at least one of: information identifying the substance contained in the cartridge, information identifying the manufacturer of the cartridge, information identifying the distributor of the cartridge and information identifying the vendor of the cartridge.

51. The cartridge of claim 49, wherein the cartridge tag comprises one of a linear barcode, a 2-D barcode and a Radio-Frequency Identification (RFID) tag.

52. The cartridge of claim 51, wherein the cartridge tag comprises one of a linear barcode and a 2-D barcode, and wherein the barcode is required for regulatory compliance.

53-75. (canceled)
